(19) United States
(12) Patent Application Publication
(10) Pub. No.: US 2025/0228693 A1
(43) Pub. Date: Jul. 17, 2025

(54) NOISE REDUCTION FOR A URINE SUCTION SYSTEM

(71) Applicant: PUREWICK CORPORATION, Covington, GA (US)

(72) Inventor: Adam Martin, Holly Springs, NC (US)

(21) Appl. No.: 18/957,011

(22) Filed: Nov. 22, 2024

Related U.S. Application Data
(63) Continuation of application No. 17/667,097, filed on Feb. 8, 2022, now Pat. No. 12,178,735.
(60) Provisional application No. 63/147,299, filed on Feb. 9, 2021.

(51) Int. Cl. A61F 5/44; A61F 5/453; A61F 5/455

(52) U.S. Cl. CPC A61F 5/4404; A61F 5/453; A61F 5/455

(57) ABSTRACT

An example urine suction device suitable for extracting urine from a urine collection device is disclosed. The suction device can include a housing, a motor disposed within the housing, a pump driven by the motor, an inlet, a speaker, and a controller. The pump can extract urine from a urine collection device. The speaker can be disposed within the housing or external to the housing. The speaker can be oriented as to attenuate noise generated by the pump, the motor, or other components of the urine suction device. The controller can cause the speaker to emit one or more first acoustic waves that at least partially attenuates one or more second acoustic waves generated by at least one of the pump or motor.

### CROSS-REFERENCE TO RELATED APPLICATIONS

This application is a continuation of U.S. application Ser. No. 17/667,097 filed on Feb. 8, 2022, which claims priority to U.S. Provisional Patent Application No. 63/147,299 filed on Feb. 9, 2021, the disclosure of each of which is incorporated herein, in its entirety, by this reference.

### BACKGROUND

In various circumstances, a person may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

In these and other circumstances, a urine collection device can be utilized to collect and retain evacuated urine. After the urine has been evacuated, the urine may need to be removed from the urine collection device. For example, a urine suction device can be used to remove urine evacuated into the urine collection device.

Devices, systems, and methods which more efficiently, hygienically, and discreetly remove or otherwise transport urine can be desirable.

### SUMMARY

Embodiments are directed to a urine suction device including a motor, a pump, a speaker, and a controller. The pump is configured to be driven by the motor. The pump is configured to extract urine from a urine collection device. The controller can be communicatively coupled to the speaker and configured to cause the speaker to emit one or more first acoustic waves that at least partially attenuates one or more second acoustic waves generated by at least one of the pump or the motor.

In another embodiment of the present disclosure, a system for transporting urine away from a user is disclosed. The system can include a urine collection device, a container, and a suction device coupled to the urine collection device. The suction device is configured to draw urine from the urine collection device into the container. The urine suction device can include a motor, a pump, a speaker, and a controller. The pump is configured to be driven by the motor. The controller can be communicatively coupled to the speaker and configured to cause the speaker to emit one or more first acoustic waves that at least partially attenuates one or more second acoustic waves generated by at least one of the pump or motor.

Embodiments are directed to a method for discretely transporting urine away from a user including supplying electrical power to a motor of a urine suction device. While electrical power is being supplied, the motor can actuate a pump of the urine suction device to transport urine away from the user. The method also includes supplying one or more electrical signals to a speaker to generate one or more first acoustic waves. The method also includes emitting the one or more first acoustic waves from the speaker. The one or more first acoustic waves can destructively interfere with one or more second acoustic waves generated by actuation of the pump.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a urine suction device coupled to a urine collection device, according to an embodiment.

FIG. 2A is an isometric view of a urine suction device and a urine collection device coupled to a user, according to an embodiment.

FIG. 2B is an isometric view of a urine suction device and a urine collection device coupled to a user, according to an embodiment.

FIG. 3A is a schematic cross-section view of a urine suction device, according to an embodiment.

FIG. 3B is a schematic cross-section view of a urine suction device, according to another embodiment.

FIG. 3C is a schematic cross-section view of a urine suction device, according to another embodiment.

FIG. 3D is a schematic cross-section view of a urine suction device, according to another embodiment.

FIG. 4 is an example flow diagram of a method for discretely transporting urine away from a user.

### DETAILED DESCRIPTION

Embodiments are directed to urine suction devices suitable for discreetly pumping urine from a urine collection device and away from the body of a person. The embodiments of the urine suction devices described herein may be fluidly coupled or otherwise attached to a urine collection device positioned in a pubic region of a user to collect urine or other fluids expelled by the user. While this disclosure primarily describes the suction device as a urine suction device, it will be appreciated that the embodiments disclosed herein can also be utilized to collect other fluids discharged from a user.

In embodiments, the urine suction device includes a housing forming or defining an interior volume. One or more components of the urine suction device can be at least partially disposed within the interior volume. For example, an electric motor and a pump can be disposed within the housing and operate to provide a suction force. In some embodiments, the housing can be omitted. While the urine suction device is operating (e.g., pumping urine from a urine collection device), the pump and/or another component of the urine suction device can generate an audible noise. This noise can be embarrassing and unflattering to the user of the urine suction device, especially while the user in a public space or has company. As such, reducing or eliminating acoustic waves generated by the urine suction device can be desirable to provide a urine suction device that is more discreet and less obtrusive.

In some embodiments, the suction device can include a housing, a motor disposed within the housing, a pump driven by the motor, an inlet, a speaker, and a controller. The pump can extract urine from a urine collection device. More specifically, the inlet can place the urine collection device and the pump in fluid communication. The speaker can be positioned within the housing and directed toward the pump to attenuate noise generated by the pump. For example, the controller can cause the speaker to emit one or more first acoustic waves that at least partially attenuates one or more second acoustic waves generated by at least one of the pump or motor. Additionally, or alternatively, the speaker can be positioned such that the acoustic waves emitted by the speaker attenuate noise emanating from an exhaust conduit of the pump.

In some embodiments, the speaker can be a first speaker of a set of speakers which are positioned within the housing and/or external to the housing. Optionally, the urine suction device can, in some embodiments, include a sensor, such as a microphone, that is communicatively coupled to the controller and detects one or more characteristics of the noise emitted from the pump and/or motor (e.g., wavelength, frequency, amplitude, etc.). The controller can thereafter generate electrical signals that cause the speaker(s) to emit acoustic waves which destructively interfere with the noise generated by the motor and/or pump.

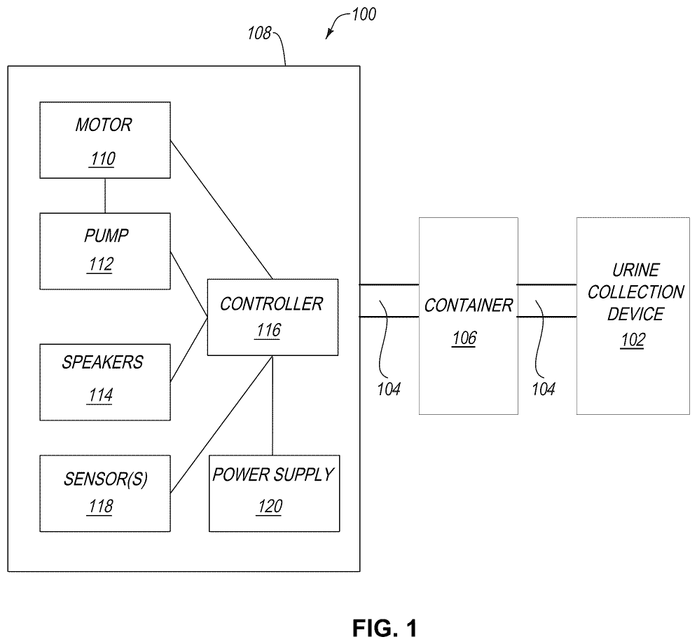

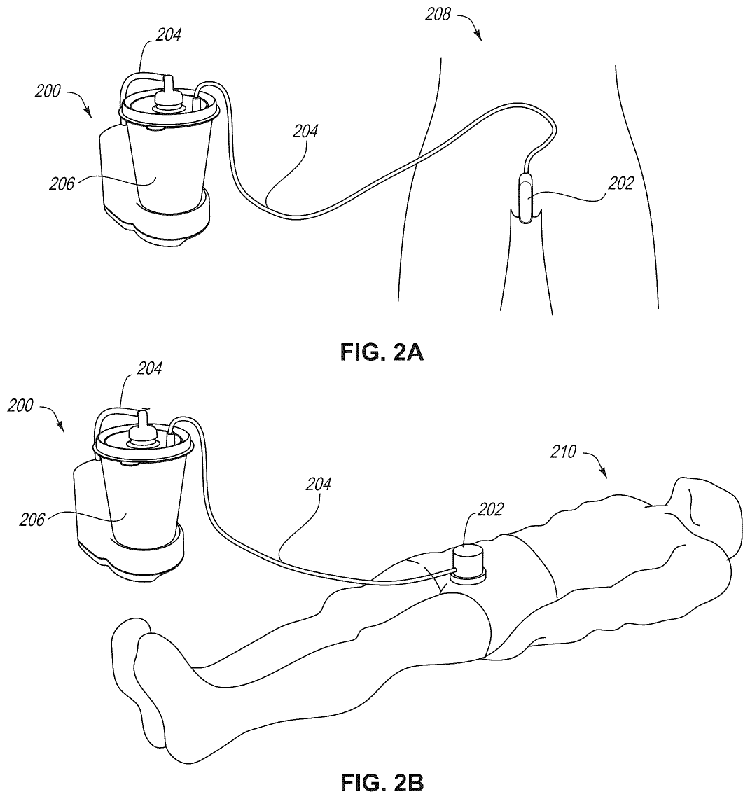

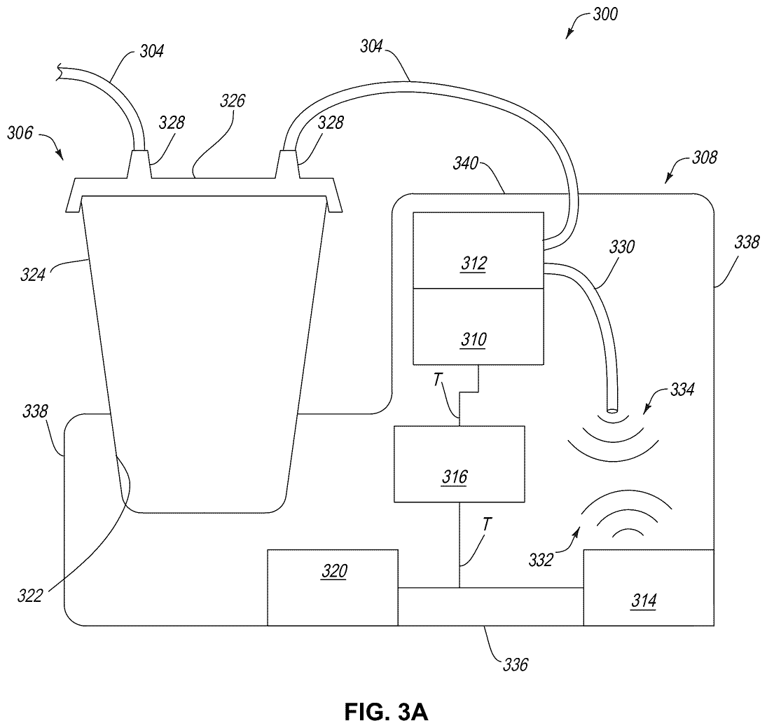

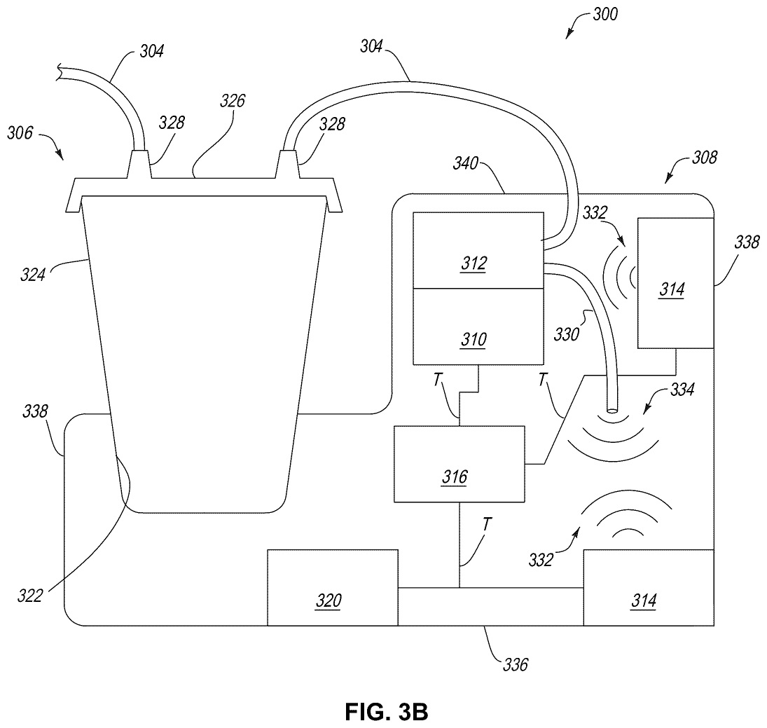

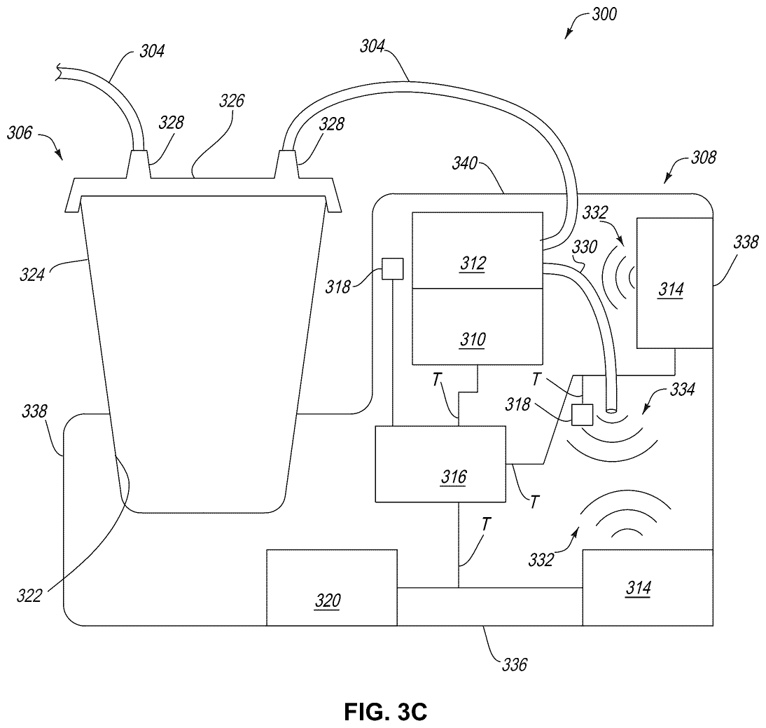

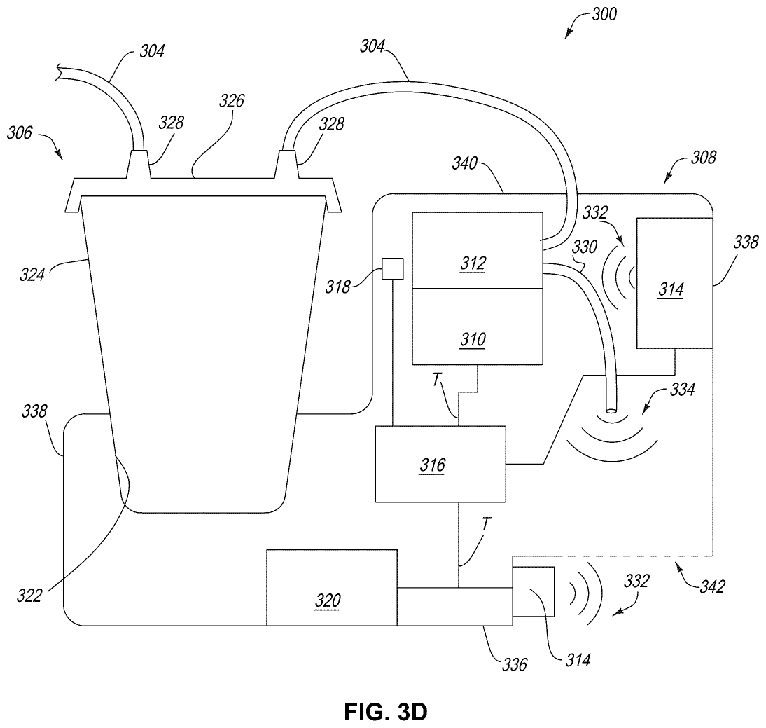

FIG. 1 shows a block diagram of a urine suction device 100 coupled to a urine collection device 102. The urine suction device 100 can be coupled to the urine collection device 102 using one or more tubes 104 and a container 106. For example, a suction force generated by the urine suction device 100 can draw urine or other fluids from the urine collection device 102, through the tube 104, and into the container 106. While this embodiment illustrates the urine suction device 100 and the container 106 as separate and distinct, the container 106 can be incorporated into or retained by the urine suction device 100 in other embodiments (as shown in FIGS. 2A-3D).

The urine suction device 100 can include a housing 108, a motor 110, a pump 112, one or more speakers 114, a controller 116, and a power supply 120. In some embodiments, the urine suction device 100 can optionally include one or more sensors 118. The housing 108 can define an internal volume and one or more of the components of the urine suction device 100 (e.g., the motor 110, the pump 112, the one or more speakers 114, the controller 116, the sensor 118, the power supply 120 etc.) can be disposed within the internal volume. For example, one or more recesses or attachment features (not shown) can be molded machined or affixed to the housing 108 to provide an attachment surface for one or more of the components of the urine suction device 100. As shown in FIGS. 2A-3D the housing 108 can also form a recess or cavity for retaining the container 106. The recess or cavity can engage a base portion of the container 106 to retain the container 106 to the urine suction device 100. The housing 108 can be assembled using one or more distinct pieces that are adhered, affixed, or otherwise assembled to form the housing 108. For example, the housing 108 can be formed of one or more molded, co-molded, machined, or stamped elements. The housing 108 can be manufactured from a polymer, ceramic, metal, a combination thereof, or any other material that can provide the functionality described herein.

While not depicted in FIG. 1, the housing 108 can support one or more input devices, such as, buttons, toggles, switches, dials, touchscreens, levers, any combination thereof, or any other input device. The one or more input devices can cause the urine collection device 102 to alter its operational state, for example, by causing the controller 116 to supply electrical power to the motor 110 to generate suction via the pump 112. The housing 108 can include various through-holes or apertures which accommodate the one or more input components. Additionally, or alternatively, the housing 108 can include various through-holes or apertures which accommodate tubes, wires, or any other fluid or electrical conduit that may need to extend into the internal volume of the housing 108. For example, an electrical cable can extend from an electrical wall outlet and through an aperture within the housing 108 to the power supply 120 to electrically power the urine suction device 100. The housing 108 will be further described below with reference to FIGS. 2A-3D.

The motor 110 can be any electrical motor capable of operating the pump 112 (i.e., causing the pump 112 to generate a negative pressure within the container 106 and/or the urine collection device 102). For example, the motor 110 can operate on alternating current (AC) or direct current (DC) and can be a brushless motor or a brushed motor. Additionally, or alternatively, the motor 110 can be DC series motor, a DC shunt motor, a stepper motor, a linear motor, a servo motor, a combination thereof, or any other motor capable of the functionality described herein. The motor 110 can drive the pump 112 to generate a suction force to draw urine out of the urine collection device 102, through the tube 104, and into the container 106. During operation (e.g., when electrical power is being supplied to the motor), the motor 110 can generate noise, such as, acoustic waves resultant of movement and vibration associated with operation. The motor 110 will be further described below with reference to FIGS. 2A-3D.

The pump 112 can be any pump capable of generating a suction force which can draw urine from the urine collection device 102. For example, the pump 112 can be a rotary lobe or gear pump, a diaphragm pump, a piston, a screw pump, a combination thereof, or any other pump capable of the functionality described herein. In some embodiments, the pump 112 can include a diaphragm (not shown) that draws air into the pump on a first stroke and exhausts air out of the diaphragm on a second stroke. The first and second strokes of the diaphragm can be actuated by rotation of the motor 110. During operation (e.g., when rotation of the motor causes actuation of the pump), the pump 112 can generate noise, such as, acoustic waves resultant of movement and vibration associated with operation. The pump 112 will be further described below with reference to FIGS. 2A-3D.

The one or more speakers 114 can emit acoustic waves, which attenuate or provide a destructive interference with acoustic waves generated by the motor, pump, and/or any other component of the urine suction device 100. For example, the one or more speakers 114 can be communicatively coupled to the controller 116 and emit one or more first acoustic waves having a maxima or amplitude that is partially or completely out of phase with a maxima or amplitude of one or more second acoustic waves generated by the motor, pump, and/or any other component of the urine suction device 100. In some embodiments, the first and second acoustic waves can be out of phase by 180 degrees or about 180 degrees. In some embodiments, the first acoustic wave, for example, can be out of phase relative to the one or more second acoustic waves by at least 140 degrees, about 140 degrees to about 155 degrees, about 155 degrees to about 170 degrees, about 170 degrees to about 185 degrees, about 185 degrees to about 200 degrees, or more than 200 degrees.

The one or more speakers 114 can include any speakers capable of emitting acoustics waves within a frequency range and amplitude which at least partially attenuate undesirable noise generated by operation of the urine suction device 100. As such, the one or more speakers 114 can include one or more full range speakers, subwoofer speakers, mid-range speakers, tweeter speakers, any other type of speaker, or a combination thereof. The one or more speakers 114 can emit one or more frequencies within a range of about 20 Hz to about 20 kHz and emit acoustic waves having amplitudes within a range of about 10 dB to about 60 dB. The one or more speakers can at least partially attenuate acoustic frequencies of about 20 Hz to about 1 kHz. The one or more speakers 114 will be further described below with reference to FIGS. 2A-3D.

In some embodiments, one or more of the components of the urine suction device 100 can be operably coupled to the controller 116. For example, the controller 116 can be disposed within the internal volume of the housing 108 and include one or more processors and memory storage having one or more operational programs stored therein. When executed by the processor, the one or more operational programs can cause the urine suction device 100 to operate. For example, an operational program can cause the one or more speakers 114 of the urine suction device 100 to emit acoustic waves while the pump 112 is operating. In some embodiments, the controller 116 can cause the speaker 114 to remain silent (e.g., a silent state) while electricity is not being supplied to the motor 110. Additionally, or alternatively, an operational program can cause the sensor 118 to detect characteristics of acoustic waves generated by the motor 110 and/or pump 112 (e.g., wavelength, frequency, amplitude, etc.).

The controller 116 can be communicatively coupled to one or more of the components of the urine suction device 100 (e.g., the motor 110, the pump 112, the one or more speakers 114, the sensor 118, the power supply 120, etc.). The controller 140 includes one or more operational programs stored therein to control one or more parameters of the urine suction device 100, such as the amplitude, frequency, phase, and other aspects of the acoustic waves emitted by the one or more speakers 114. The one or more operational programs include machine readable and executable instructions to control operation of the urine suction device 100. The controller 116 will be further described below with reference to FIGS. 2A-3D.

The sensor 118 can be a single sensor or multiple sensors disposed in various locations relative to the housing 108. The sensor 118 can be communicatively coupled to the controller 116 and supply electrical signals to the controller 116 that are used to generate the acoustic waves emitted by the one or more speakers 114. In some embodiments, the sensor 118 can be a microphone that detects or measures characteristics of the acoustic waves generated by the motor 110 and/or the pump 112. For example, the sensor 118 can be disposed adjacent an exhaust conduit or outlet of the pump 112 and detect characteristics of the acoustic waves emitting from the exhaust conduit of the pump 112. The sensor 118 can be one or more of a micro-electro-mechanical system (MEMS) microphone, a condenser-based microphone, a dynamic microphone, piezoelectric-based microphone, any other microphone, or a combination thereof.

In some embodiments, the controller 116 can analyze the signals delivered by the sensor 118 and thereafter generate and deliver electrical signals to the one or more speakers 114. For example, the controller 116 can include software and/or firmware which supports signal processing techniques, such as, analog or digital signal processing. The signals delivered to the speaker 114 can be processed by the controller 116 to derive alternative signals that cause the speakers to output acoustic waves which destructively interfere with the acoustic waves produced by operation of the pump 112 and/or motor 110. The sensor 118 will be further described below with reference to FIGS. 2A-3D.

The power supply 120 may be operably coupled to the controller 116, the motor 110, the pump 112, the one or more speakers 114, the sensor 118, or any other components of the urine suction device 100 to provide electrical power to the urine suction device 100. For example, the power supply 120 may include one or more batteries (e.g. lithium-ion, nickel-cadmium, nickel-metal hydride, etc.) or portable chargers (e.g., power banks). The one or more batteries may be rechargeable. In examples, the one or more batteries may be modular battery packs, which may be removed and replaced. In examples, the one or more batteries have a connection for charging, such as a connection for the portable charger. The power supply 120 may be a replaceable and rechargeable battery, such as a 12 volt battery. The rechargeable battery may be a lithium ion battery, lithium-ion polymer, a nickel-cadmium battery, nickel-metal hydride, lead acid, etc., batteries. The power supply 120 may include a plurality of rechargeable batteries. The rechargeable battery may be at least a 1 volt battery, such as 1.5 volts to 3 volts, 3 volts to 6 volts, 6 volts to 9 volts, 9 volts to 12 volts, 12 volts to 15 volts, 15 volts to 24 volts, greater than 12 volts, less than 24 volts, or less than 15 volts.

In examples, the power supply 120 may include a cord or wired connection for connecting to a power outlet. For example, the power supply 120 may include 110 volt, 220 volt, or similar connections. The cord may allow the user to plug the urine suction device 100 into a power outlet in a room, an extension cord, or a power station or power bank (e.g., battery pack or bank). Accordingly, the power supply 120 may include a wall outlet, the extension cord, or a power station or power bank. In examples, the power supply 120 may include both a wired connection for coupling to a power source and a battery pack. Accordingly, the urine suction device 100 may be run with our without battery power. In examples, the wired connection may be provided as a detachable power cord which may be removed from the urine suction device 100. The wired connection may serve to recharge the battery pack and provide power to the urine suction device 100.

The container 106 can be distinct from the urine suction device 100 or releasably retained on the housing 108 of the urine collection device 102 (as shown in FIGS. 2A-3D). The container 106 can be any container capable of storing urine or other fluids that are extracted from the urine collection device 102 by a suction force applied by the urine suction device 100. As such, the container 106 can be manufactured of any rigid or flexible material capable of reliably retaining fluid. The container 106 can be machined, molded, co-molded, or otherwise manufactured from any material, such as, a polymer, a metal, a ceramic, or a combination thereof. The container 106 will be further described below with reference to FIGS. 2A-3D.

The urine collection device 102 can be any urine collection device presently available or otherwise developed in the future. The urine collection device 102 may be adapted to extract urine or other fluids discharged by female and/or male persons. One suitable non-limiting example of a urine collection device that can be used is an external catheter, available from PureWick, Inc. Any urine collection device capable of collecting discharged urine from a male or female user can be utilized with the present disclosure. For example, the urine collection devices described in U.S. Patent Publication No. 2019/0282391 filed 6 Jun. 2019 and U.S. Pat. No. 10,390,989 filed 8 Sep. 2016, the disclosures of which are incorporated herein, in their entirety, by this reference.

FIGS. 2A and 2B show isometric views of a urine suction device 200 and a urine collection device 202, according to an embodiment. More specifically, a first tube 204 extends from the urine suction device 200 to a container 206 and a second tube 204 extends from the container 206 to the urine collection device 202. The urine collection device 202 can be disposed on a female user 208 (as shown in FIG. 2A) or a male user 210 (as shown in FIG. 2B). On female users, the urine collection device 202 can be placed between the legs or labia and held snugly against the external urethra by the pressure of friction from the user's body, by the pressure of the legs or by such means as an undergarment, elastic strips, and/or adhesive tape. In male applications, the urine collection device 202 can be positioned around the penis. The urine suction device 200 can generate a negative pressure within the container 206 to draw or extract urine from the urine collection device 202 into the container 206.

FIGS. 3A-3D show various non-limiting examples of a urine suction device 300, according to an embodiment. Features from any of the embodiments described herein may be used in combination with one another, without limitation. FIG. 3A shows a schematic cross-section view of the urine suction device 300, according to some embodiments. The urine suction device 300 can be substantially similar to and can include some or all of the features of the urine suction devices 100 and 200. For example, one or more tubes 304 can place the urine suction device 300 in fluid communication with a container 306 and/or a urine collection device (not shown).

The urine suction device 300 can include a housing 308, a motor 310, a pump 312, a speaker 314, a controller 316, a sensor 318, and a power supply 320. The motor 310 can be substantially similar to and can include some or all of the features of the motor 110 described with reference to FIG. 1. For example, the motor 310 can drive the pump 312 to generate a suction force to draw urine out of the urine collection device (not shown), through the tube 304, and into the container 306. The pump 312 can be substantially similar to and can include some or all of the features of the pump 112 described with reference to FIG. 1. For example, the pump 312 can be a rotary lobe or gear pump, a diaphragm pump, a piston, a screw pump, a combination thereof, or any other pump capable of the functionality described herein. The controller 316 can be substantially similar to and can include some or all of the features of the controller 116 described with reference to FIG. 1. For example, the controller 316 can be communicatively coupled to one or more of the components of the urine suction device 300 (e.g., the motor 310, the pump 312, the one or more speakers 314, a sensor 318, the power supply 320, etc.) through electrical traces T. The power supply 320 can be substantially similar to and can include some or all of the features of the power supply 120 described with reference to FIG. 1. For example, the power supply 320 may be operably coupled to the controller 316, the motor 310, the pump 312, the one or more speakers 314, the sensor 318, or any other components of the urine suction device 300 to provide electrical power to the urine suction device 300.

The housing 308 can be substantially similar to and can include some or all of the features of the housing 108 described with reference to FIG. 1. While the housing 308 is illustrated as having a particular cross-sectional shape, those skilled in the art will appreciate that the cross-sectional shape illustrated in FIGS. 3A-3D merely represents one example cross-sectional shape of many. Similarly, while the components of the urine suction device 300 (e.g., the motor 310, the pump 312, the speaker 314, the controller 316, and the power supply 320, etc.) are illustrated in respective positions within the housing 308, those skilled in the art will readily appreciate that one or more of the components can be positioned elsewhere within the housing 308 or external to the housing 308. The housing 308 can form a cavity or recess 322 for retaining the container 306. The cavity or recess 322 can have a profile which substantially matches a base portion of the container 306 such that base portion of the container 306 is reliably retained within the recess 322.

The container 306 can include a body 324 and a lid 326. The body 324 can retain or store a quantity of fluid, such as, urine or other fluid which is extracted or pumped from a urine collection device (not shown). The lid 326 can be affixed to the body 324, for example, by a threaded engagement, a friction fit engagement, a protrusion interlocking engagement, or any other mechanism capable of retaining the lid 326 to the body 324. The lid 326 can include one or more inlets 328 which receive the respective tubes 304 or other fluid conduits and place the container 306 in fluid communication with a urine collection device (not shown) and/or the urine suction device 300. The one or more inlets 328 can form respective apertures within the lid 326 to place the container 306 in fluid communication with a urine collection device (not shown) and/or the urine suction device 300. While the one or more inlets 328 are illustrated as being formed on the lid 326, those having skill in the art will readily appreciate that one or more inlets 328 can additionally, or alternatively, be formed on the body 324 of the container 306.

In some embodiments, the pump 312 can intake fluid (e.g., air) through the tube 304 and exhaust the fluid through an exhaust conduit 330. In other words, while the pump 312 is being driven by the motor 310, fluid (e.g., air) can be drawn or sucked into the pump 312 and exhausted through the exhaust conduit 330. This process can generate audible acoustic waves which signal that urine or other fluid is currently being drawn or extracted from the urine collection device. For example, operation of the pump 312 (e.g., oscillating a diaphragm within the pump 312) and exhausting fluid through the exhaust conduit 330 can generate acoustic waves which are detectable by persons located near the urine suction device 300.

The one or more speakers 314 can emit one or more acoustic waves which at least partially attenuate the acoustic waves generated by the motor 310, the pump 312, or a combination thereof. In other words, one or more first acoustic waves emitted by the speaker 314 can destructively interfere with one or more second acoustic waves generated by the motor 310 and/or pump 312 to reduce or diminish the amplitude of the resultant combined acoustic waves as to render the one or more second acoustic waves less discernable by persons near the urine suction device 300. The one or more first acoustic waves can have a first frequency that is similar or identical to a second frequency of the second acoustic wave, however, the first and second frequencies can be out of phase from one another (e.g., 180 degrees out of phase). The one or more first acoustic waves can attenuate or diminish at least 40%, about 40% to about 60%, about 60% to about 80%, or more than 80% of the second acoustic wave. In some embodiments, the first one or more acoustic waves can attenuate or diminish the one or more second acoustic waves by at least 1 dB, about 1 dB to about 5 dB, about 5 dB to about 10 dB, or more than 10 dB.

In some embodiments, as shown in FIG. 3A, the speaker 314 is disposed within an internal volume of the housing 308 and directed toward the exhaust conduit 330 such that acoustic wave(s) 332 emitted by the speaker 314 at least partially attenuate or diminish acoustic wave(s) 334 propagating from the exhaust conduit 330 of the pump 312. While the speaker 314 is illustrated as positioned at a base 336 of the housing 308, the speaker 314 may be additionally, or alternatively, positioned on a side wall 338 or ceiling 340 of the housing 308 in some embodiments. The side wall 338 can extend around a periphery of the housing 308 and extend between the base 336 and the ceiling 340.

In some embodiments, as shown in FIG. 3B, multiple speakers 314 are disposed within the internal volume of the housing 308 and respectively directed toward the exhaust conduit 330, the motor 310, and the pump 312, such that acoustic wave(s) 332 emitted by the multiple speakers 314 at least partially attenuate or diminish acoustic wave(s) 334 propagating from the exhaust conduit 330, the motor 310, and the pump 312. Each of the multiple speakers 314 can be directed or oriented to emit the acoustic waves 332 in a direction that is perpendicular, oblique, or parallel relative to acoustic waves 334 generated by the motor 310, the pump 312, or the exhaust conduit 330. In other words, each of the multiple speakers 314 can be positioned and oriented within the housing 308 to most efficiently diminish or attenuate noise generated by the motor 310, the pump 312, the exhaust conduit 330, or a combination thereof. For example, the speaker 314 disposed on the base 336 of the housing 308 can be oriented to emit acoustic waves 332 toward the motor 310 and pump 312 in a first direction while the speaker 314 disposed on the side wall 338 of the housing 308 can be oriented to emit acoustic waves 332 toward the motor 310 and the pump 312 in a second direction. While the first and second directions are illustrated as perpendicular to one another in FIG. 3B, the first and second directions can be perpendicular, parallel, or oblique to one another in other embodiments.

In some examples, each individual speaker of the multiple speakers 314 can emit acoustic waves 332 having distinct characteristics (e.g., wavelength, frequency, amplitude, etc.) from the characteristics of acoustic waves 332 emitted by the other speaker(s) of the multiple speakers 314. For example, the speaker 314 coupled to the side wall 338 of the housing 308 can emit acoustic waves having at least one characteristic that is different than the at least one characteristic of acoustic waves emitted by the speaker 314 coupled to the base 336 of the housing 308. In some examples, two or more of the multiple speakers can emit acoustic waves 332 having similar or identical characteristics (e.g., wavelength, frequency, amplitude, etc.).

In some embodiments, as shown in FIG. 3C, multiple speakers 314 and one or more sensors 318 can be disposed within an internal volume of the housing 308. The multiple speakers 314 can emit acoustic wave(s) 332 to at least partially attenuate or diminish acoustic wave(s) 334 propagating from the motor 310, the pump 312, and/or the exhaust conduit 330 of the pump 312. Each of the one or more sensors 318 can be communicatively coupled to the controller 316 and supply electrical signals to the controller 316. The controller 316 can analyze the electrical signals and generate the acoustic waves 332 emitted by the multiple speakers 314. For example, one of the sensors 318 can be disposed adjacent an exhaust conduit 330 and detect characteristics of the acoustic waves 334 emitting from the exhaust conduit 330. In some embodiments, one or more of the sensors 318 can be a microphone that detects or measures characteristics of the acoustic waves 334 generated by the motor 310 and/or the pump 312 and delivers the measurements to the controller 316. Thereafter, the controller 316 can determine the characteristics of the acoustic waves 334 and cause the speakers 314 to emit acoustic waves 332 that most effectively attenuate or diminish the acoustic waves 334. While the sensors 318 are illustrated as being respectively positioned near the pump 312 and near the exhaust conduit 330 in FIG. 3C, some embodiments can include sensors disposed at other locations within or external to the housing 308.

In some embodiments, as shown in FIG. 3D, the speaker 314 can be disposed external to the housing 308 and directed toward a vent 342 such that acoustic wave(s) 332 emitted by the speaker 314 at least partially attenuate or diminish acoustic wave(s) 334 propagating from the vent 342. The vent 342 can be a series of apertures, slots, perforations, a combination thereof, or any other through-hole which places the internal volume of the housing 308 in fluid communication with an environment external to the housing 308. For example, the vent 342 can be a set of slots which enable air or other fluid exhausted from the pump 312 to exit the internal volume of the housing 308. Additionally, or alternatively, the vent 342 may provide a fluid path which enables heat generated by one or more components within the urine suction device 300 (e.g., the motor 310, the pump 312, the controller 316, etc.) to vent through the housing 308.

The motor 310, the pump 312, and/or other components of the urine suction device 300 can generate noise (e.g., the one or more second acoustic waves 334) which propagates within the internal volume of the housing 308 and through the vent 342 into the environment external to the housing 308. One or more speakers 314 can be disposed external to the housing 308 to emit one or more acoustic waves which at least partially attenuate or diminish the acoustic waves generated by the motor 310, the pump 312, and/or other components of the urine suction device 300. For example, as shown in FIG. 3D, the speaker 314 can be disposed adjacent to the vent 342 and oriented to emit the one or more first acoustic waves 332 such that the one or more first acoustic waves 332 propagate or travel parallel to the vent 342. While the speaker 314 in FIG. 3D is oriented to emit the one or more first acoustic waves 332 parallel to the vent 342, the speaker 314 can be additionally, or alternatively, be oriented such that the one or more first acoustic waves 332 propagate or travel perpendicular or oblique to the vent 342. In other words, the one or more speakers 314 can be oriented to emit acoustic waves parallel, perpendicular, or oblique to at least one of the base 336, the side wall 338, or the ceiling 340 of the housing 308.

While the example illustrated in FIG. 3D shows a single speaker 314 disposed external to the housing 308, other examples can include an additional, or alternative, speaker 314 disposed within the housing 308 and oriented to emit acoustic waves toward the vent 342. In some examples, the urine suction device 300 can include at least one speaker 314 disposed within the housing 308 as well at least one speaker 314 positioned external to the housing 308. While the example illustrated in FIG. 3D shows the vent 342 formed or otherwise disposed within the base 336 of the housing, other examples can include additional, or alternative, vents 342 disposed within the base 336, the side wall 338, or the ceiling 340.

The one or more speakers 314 can be communicatively coupled to the controller 316 through a wired communication path or a wireless communication path. For example, a cable can interconnect the speakers 314 and the controller 316 or a wireless protocol such as IEEE 802 (i.e., Bluetooth and Wi-Fi wireless networking technologies) can be utilized to connect the speakers 314 to the controller 316. In some embodiments, one or all of the speakers 314 can be repositionable within or outside of the housing 308. For example, one or more of the speakers 314 can be positioned some distance away from the housing 308 yet oriented toward the housing 308 such that the acoustic waves 332 emitted by the speaker 314 destructively interfere with any acoustic waves 334 emanating from the housing 308.

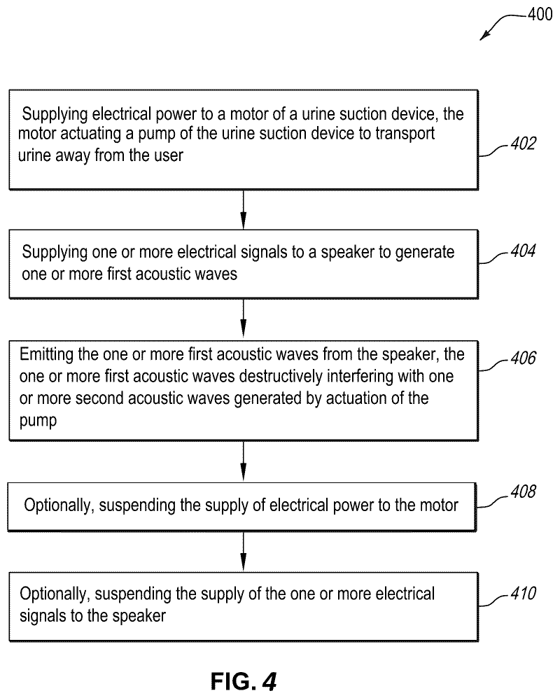

FIG. 4 illustrates an example method 400 for discretely transporting urine away from a user using a suction device. The suction device can be substantially similar to, and can include some or all of the features and/or components of the suction devices described herein, such as suction devices 100, 200, 300. For example, the suction device can include a motor, a pump, one or more speakers, a controller, a power supply, one or more sensors, and/or any other component of the other suction devices disclosed herein.

The method 400 includes the act 402 of supplying electrical power to a motor of a urine suction device, the motor actuating a pump of the urine suction device to transport urine away from the user. The method 400 includes the act 404 of supplying one or more electrical signals to a speaker to generate one or more first acoustic waves. The method 400 includes the act 406 of emitting the one or more first acoustic waves from the speaker, the one or more first acoustic waves destructively interfering with one or more second acoustic waves generated by actuation of the pump. Optionally, the method 400 can include the act 408 of suspending the supply of electrical power to the motor. Optionally, the method 400 can include the act 410 of suspending the supply of the one or more electrical signals to the speaker.

Accordingly, the method 400 can be utilized to discretely transport urine away from a user using a suction device by mitigating or reducing noise generated by operation of the motor and/or pump within the suction device. The method 400 can include more or fewer acts than the acts 402-410. For example, the method 400 may optionally include one or more acts of measuring an acoustic characteristic of the one or more second acoustic waves using a sensor of the urine suction device; analyzing the acoustic characteristic using a controller of the urine suction device; and generating, using the controller, the one or more electrical signals such that the one or more first acoustic waves is out of phase relative to the one or more second acoustic waves. In other words, some of the acts are optional and therefore need not be implemented to discretely transporting urine away from a user using a suction device.

The method 400 includes the act 402 of supplying electrical power to a motor of a urine suction device, the motor actuating a pump of the urine suction device to transport urine away from the user. In some embodiments, the electrical power can be supplied directly to the motor from a power supply. Alternatively, or additionally, the electrical power can be supplied to the motor through a controller coupled to the power supply. The motor can be substantially similar to, and can include some or all of the features and/or components of the motors described herein, such as motor 110, 310. For example, the motor can operate on alternating current (AC) or direct current (DC) and can be a brushless motor or a brushed motor. Additionally, or alternatively, the motor can be DC series motor, a DC shunt motor, a stepper motor, a linear motor, a servo motor, a combination thereof, or any other motor capable of the functionality described herein. Similarly, the pump can be substantially similar to, and can include some or all of the features and/or components of the pumps described herein, such as pumps 112, 312. For example, the pump can be a rotary lobe or gear pump, a diaphragm pump, a piston, a screw pump, a combination thereof, or any other pump capable of the functionality described herein.

The method 400 includes the act 404 of supplying one or more electrical signals to a speaker to generate one or more first acoustic waves. In embodiments, the controller can supply the electrical signals to the speaker which generate the one or more first acoustic waves. The speaker can be substantially similar to, and can include some or all of the features and/or components of the speakers described herein, such as speaker 114, 314. For example, the speaker can be one or more speakers including one or more full range speakers, subwoofer speakers, mid-range speakers, tweeter speakers, any other type of speaker, or a combination thereof. In some embodiments, the electrical power can be supplied to the motor at the same time (i.e., simultaneously) as the electrical signals are supplied to the speaker. Alternatively, the electrical power to the motor can be supplied before or after the electrical signals are supplied to the speaker.

The method 400 includes the act 406 of emitting the one or more first acoustic waves from the speaker, the one or more first acoustic waves destructively interfering with one or more second acoustic waves generated by actuation of the pump. For example, the speaker can be directed or oriented to emit the acoustic waves in a direction that is perpendicular, oblique, or parallel relative to the second acoustic waves generated by the motor and/or the pump. In other words, the speaker can be positioned and oriented to most efficiently diminish or attenuate noise generated by the motor, the pump, or a combination thereof. The one or more first acoustic waves can destructively interfere with the one or more second acoustic waves, for example, by having a maxima or amplitude that is partially or completely out of phase with a maxima or amplitude of one or more second acoustic waves.

Optionally, the method 400 can include the act 408 of suspending the supply of electrical power to the motor. Optionally, the method 400 can include the act 410 of suspending the supply of the one or more electrical signals to the speaker. In some embodiments, the suspension of electrical power to the motor can occur simultaneously with the suspension of electrical signals to the speaker. Alternatively, the electrical power to the motor can be suspended before or after the electrical signals supplied to the speaker are suspended.

In some embodiments, the method 400 can include the acts of measuring an acoustic characteristic of the one or more second acoustic waves using a sensor of the urine suction device; analyzing the acoustic characteristic using a controller of the urine suction device; and generating, using the controller, the one or more electrical signals such that the one or more first acoustic waves is out of phase relative to the one or more second acoustic waves. The acoustic characteristic can be any property or attribute of the one or more second acoustic waves, for example, wavelengths, frequencies, amplitudes, etc. The controller can include software and/or firmware which supports signal processing techniques, such as, analog or digital signal processing to analyze the acoustic characteristic.

While various embodiments of the urine suction systems and devices have been described above, it should be understood that they have been presented by way of example only, and not limitation. Those of ordinary skill in the art having the benefit of this disclosure would recognize that the ordering of certain steps may be modified and such modifications are in accordance with the variations of the invention. Additionally, certain of the steps may be performed concurrently in a parallel method when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

1. A urine suction device, comprising:
a housing;
a urine extraction system disposed at least partially within the housing and configured to extract urine from a urine collection device; and
a speaker configured to at least partially attenuate sound generated by the urine extraction system.

2. The urine suction device of claim 1, wherein the speaker is disposed at least partially within the housing and oriented towards the urine extraction system.

3. The urine suction device of claim 2, wherein the urine extraction system includes a motor disposed at least partially in the housing and a pump disposed at least partially in the housing, the pump being configured to be driven by the motor and extract urine from the urine collection device and the speaker being oriented towards the pump and the motor.

4. The urine suction device of claim 3, further comprising:
wherein the urine extraction system further includes an exhaust conduit extending from the pump, the pump being configured to exhaust fluid through the exhaust conduit;
an additional speaker oriented and configured to attenuate sound from the exhaust conduit.

5. The urine suction device of claim 1, further comprising a controller communicatively coupled to the speaker and configured to cause the speaker to at least partially attenuate the sound generated by the urine extraction system.

6. The urine suction device of claim 5, further comprising a sensor communicatively coupled to the controller and configured to detect characteristics acoustic waves emitted by the urine extraction system and direct the speaker to emit acoustic waves at least partially based on the characteristic acoustic waves emitted by the urine extraction system detected by the sensor effective to at least partially attenuate the sound generated by the urine extraction system.

7. The urine suction device of claim 5, wherein:
the urine extraction system is communicatively coupled to the controller and the controller is configured to simultaneously activate the urine extract system and the speaker; and
the controller is configured to silence the speaker when electricity is not being supplied to the urine extraction system.

8. A system for transporting urine away from a user, the system comprising:
a urine collection device;
a container; and
a suction device coupled to the urine collection device and configured to draw urine from the urine collection device into the container, the suction device comprising:
a housing;
a urine extraction system disposed at least partially within the housing and configured to extract urine from a urine collection device; and
a speaker configured to at least partially attenuate sound generated by the urine extraction system.

9. The system of claim 8, wherein the speaker is disposed at least partially within the housing and oriented towards the urine extraction system.

10. The system of claim 9, wherein the urine extraction system includes a motor disposed at least partially in the housing and a pump disposed at least partially in the housing, the pump being configured to be driven by the motor and extract urine from the urine collection device and the speaker being oriented towards the pump and the motor.

11. The system of claim 10, further comprising:
wherein the urine extraction system further includes an exhaust conduit extending from the pump, the pump being configured to exhaust fluid through the exhaust conduit;
an additional speaker oriented and configured to attenuate sound from the exhaust conduit.

12. The system of claim 8, further comprising a controller communicatively coupled to the speaker and configured to cause the speaker to at least partially attenuate the sound generated by the urine extraction system.

13. The system of claim 12, further comprising a sensor communicatively coupled to the controller and configured to detect characteristics acoustic waves emitted by the urine extraction system and direct the speaker to emit acoustic waves at least partially based on the characteristic acoustic waves emitted by the urine extraction system effective detected by the sensor to at least partially attenuate the sound generated by the urine extraction system.

14. The system of claim 12, wherein:
the urine extraction system is communicatively coupled to the controller and the controller is configured to simultaneously activate the urine extract system and the speaker; and
the controller is configured to silence the speaker when electricity is not being supplied to the urine extraction system.

15. A method for discretely transporting urine away from a user, the method comprising:
actuating a urine extraction system of a suction device effective to transport the urine away from the user, the urine extraction system disposed at least partially in a housing of the suction device; and
actuating a speaker effective to at least partially attenuate sound generated by the urine extraction system as the urine extraction system transports the urine away from the user.

16. The method of claim 15, wherein:
actuating the urine extraction system includes actuating a motor of the urine extraction system effective to actuate a pump of the urine extraction system to transport the urine away from the user; and
actuating the speaker includes actuating the speaker disposed at least partially within housing and oriented towards the pump and the motor effective to at least partially attenuate the sound generated by the pump and the motor as the urine extraction system transports the urine away from the user.

17. The method of claim 16, further comprising actuating an additional speaker disposed at least partially within the housing effective to attenuate sound from an exhaust conduit exhausting fluid from the pump.

18. The system of claim 15, wherein actuating the speaker includes causing the speaker to at least partially attenuate the sound generated by the urine extraction system with a controller communicatively coupled to the speaker.

19. The method of claim 18, further comprising:
detecting characteristics acoustic waves emitted by the urine extraction system with a sensor communicatively coupled to the controller;
wherein causing the speaker to at least partially attenuate the sound generated by the urine extraction system with the controller communicatively coupled to the speaker includes directing the speaker to emit acoustic waves at least partially based the characteristics acoustic waves detected by the sensor effective to at least partially attenuate the sound generated by the urine extraction system.

20. The system of claim 18, further comprising:
simultaneously actuating the urine extraction system and the speaker with the controller;
silencing the speaker with the controller when the urine extraction system is not actuated.
